(19) United States
(12) Patent Application Publication
(10) Pub. No.: US 2005/0177238 A1
(43) Pub. Date: Aug. 11, 2005

(54) Radiolucent bone graft

(76) Inventors: Ashok C. Khandkar, Salt Lake City, UT (US); Bret M. Berry, Sandy, UT (US); Darrel S. Brodke, Salt Lake City, UT (US); Ramaswamy Lakshminarayanan, Salt Lake City, UT (US); Mahendra S. Rao, Timonium, MD (US)

Correspondence Address: KELLY LOWRY & KELLEY, LLP, 6320 CANOGA AVENUE, SUITE 1650, WOODLAND HILLS, CA, 91367, US

(21) Appl. No.: 11/040,477

(22) Filed: Jan. 20, 2005

Related U.S. Application Data
(63) Continuation-in-part of application No. 10/137,106, filed on Apr. 30, 2002, now Pat. No. 6,846,327.
(60) Provisional application No. 60/287,824, filed on May 1, 2001.

(51) Int. Cl. A61F 2/44; A61F 2/28

(52) U.S. Cl. 623/17.11; 623/23.51; 623/23.57

(57) ABSTRACT

An improved bone graft is provided for human implantation, bone graft includes a substrate block of high strength biocompatible material having a selected size and shape to fit the anatomical space, and a controlled porosity analogous to natural bone. The substrate block may be coated with a bio-active surface coating material such as hydroxyapatite or a calcium phosphate to promote bone ingrowth and enhanced bone fusion. Upon implantation, the bone graft provides a spacer element having a desired combination of mechanical strength together with osteoconductivity and osteoinductivity to promote bone ingrowth and fusion, as well as radiolucency for facilitated post-operative monitoring. The bone graft may additionally carry one or more natural or synthetic therapeutic agents for further promoting bone ingrowth and fusion.

### BACKGROUND OF THE INVENTION

This application is a continuation-in-part of copending U.S. Ser. No. 10/137,106, filed Apr. 30, 2002, which in turn claims the benefit of U.S. Provisional Application No. 60/287,824, filed May 1, 2001.

This invention relates generally to improvements in bone grafts such as spinal fusion cages of the type designed for human implantation between adjacent spinal vertebrae, to maintain the vertebrae in substantially fixed spaced relation while promoting interbody bone ingrowth and fusion therebetween. More particularly, this invention relates to an implantable bone graft having an improved combination of enhanced mechanical strength together with osteoinductive and osteoconductive properties, in a device that additionally and beneficially provides visualization of bone growth for facilitated post-operative monitoring.

Implantable bone grafts are known in the art and are routinely used by orthopedic surgeons to keep skeletal structures in a desired spaced-apart relation while bone ingrowth and fusion takes place. Such grafts are also used to provide weight bearing support between adjacent skeletal bodies and thus correct clinical problems. Such grafts are indicated for surgical treatment to reinforce weak bony tissue. These conditions have been treated by using constructs, typically made from metals such as titanium or cobalt chrome alloys such as used in orthopedic implants, and allograft (donor) or autograft (patient) bone to promote bone ingrowth and fusion.

Typical bone grafts, such as plugs for example, have hollow or open spaces that are usually filled with bone graft material, either autogenous bone material provided by the patient or allogenous bone material provided by a third party donor. These devices also have lateral slots or openings which are primarily used to promote ingrowth of blood supply and grow active and live bone. These implants may also have a patterned exterior surface such as a ribbed or serrated surface or a screw thread to achieve enhanced mechanical interlock between skeletal structures, with minimal risk of implant dislodgement from the site. See, for example, U.S. Pat. Nos. 5,785,710; and 5,702,453. Typical materials of construction for such devices include bio-compatible carbon fiber reinforced polymers, cobalt chrome alloys, and stainless steels or titanium alloys. See, for example, U.S. Pat. No. 5,425,772.

Most state-of-the-art bone grafts are made from titanium alloy and allograft (donor) bone, and have enjoyed clinical success as well as rapid and widespread use due to improved patient outcomes. However, traditional titanium-based implant devices exhibit poor radiolucency characteristics, presenting difficulties in post-operative monitoring and evaluation of the fusion process due to the radio-shadow produced by the non-lucent metal. There is also clinical evidence of bone subsidence and collapse which is believed to be attributable to mechanical incompatibility between natural bone and the metal implant material. Moreover, traditional titanium-based implant devices are primarily load bearing but are not osteoconductive, i.e., not conducive to direct and strong mechanical attachment to patient bone tissue, leading to potential graft necrosis, poor fusion and stability. By contrast, allograft bone implants exhibit good osteoconductive properties, but can subside over time as they assimilate into natural bone. Further, they suffer from poor pull out strength resulting in poor stability, primarily due to the limited options in machining the contact surfaces. Allograft bone implants also have variable materials properties and, perhaps most important of all, are in very limited supply. A small but finite risk of disease transmission with allograft bone is a factor as well. In response to these problems some developers are attempting to use porous tantalum-based metal constructs, but these have met with limited success owing to the poor elastic modulii of porous metals.

A typical titanium alloy bone graft device is constructed from a hollow cylindrical and threaded metal cage-like construct with fenestrations that allow communication of the cancellous host tissue with the hollow core, which is packed with morselized bone graft material. This design, constrained by the materials properties of titanium alloys, relies on bony ingrowth into the fenestrations induced by the bone graft material. However, the titanium-based structure can form a thin fibrous layer at the bone/metal interface, which degrades bone attachment to the metal. In addition, the hollow core into which the graft material is packed may have sub-optimal stress transmission and vascularization, thus eventually leading to failure to incorporate the graft. Mechanical stability, transmission of fluid stress, and the presence of osteoinductive agents are required to stimulate the ingrowth of vascular buds and proliferate mesenchymal cells from the cancellous host tissue into the graft material. However, most titanium-based bone graft devices in use today have end caps or lateral solid walls to prevent egress of the graft outwardly from the core and ingress of remnant disc tissue and fibroblasts into the core.

Autologous (patient) bone fusion has been used in the past and has a theoretically ideal mix of osteoconductive and osteoinductive properties. However, supply of autologous bone material is limited and significant complications are known to occur from bone harvesting. Moreover, the costs associated with harvesting autograft bone material are high, requiring two separate incisions, with the patient having to undergo more pain and recuperation due to the harvesting and implantation processes. Additionally, autologous cancellous bone material has inadequate mechanical strength to support musculoskeletal forces by itself, whereby the bone material is normally incorporated with a metal-based construct.

Ceramic materials provide potential alternative structures for use in spinal fusion implant devices. In this regard, monolithic ceramic constructs have been proposed, formed from conventional materials such as hydroxyapatitie (HAP) and/or tricalcium phosphate (TCP). See, for example, U.S. Pat. No. 6,037,519. However, while these ceramic materials may provide satisfactory osteoconductive and osteoinductive properties, they have not provided the mechanical strength necessary for the implant.

Thus, a significant need exists for further improvements in and to the design of bone grafts, particularly to provide a high strength implant having high bone ingrowth and fusion characteristics, together with substantial radiolucency for effective and facilitated post-operative monitoring.

Hence, it is an object of the present invention to provide an improved bone graft made from a bio-compatible open pore structure, which has a radiolucency similar to that of the surrounding bone. It is also an object of the present invention to provide a substrate of adequate bio-mechanical strength for carrying biological agents which promote bone ingrowth, healing and fusion. It is a further objective of the present invention to provide a fusion device which has mechanical properties that substantially match that of natural bone.

### SUMMARY OF THE INVENTION

In accordance with the invention, an improved bone graft is provided for human implantation into the space between a pair of adjacent skeletal structures, to maintain the adjacent skeletal anatomy in a predetermined and substantially fixed spaced relation while promoting bone ingrowth and fusion. In this regard, the improved bone graft of the present invention is designed for use in addressing clinical problems indicated by surgical treatment of bone fractures, skeletal non-unions, weak bony tissue, degenerative disc disease, discogenic lower back pain, and spondylolisthesis.

The improved bone graft comprises a substrate block formed from a bio-compatible material composition having a relatively high bio-mechanical strength and load bearing capacity, substantially equivalent to natural cortical bone. This substrate may be porous, open-celled, or dense solid. A preferred material of the high strength substrate block comprises a ceramic material. The substrate block may be porous, having a porosity of about 10% to about 80% by volume with open pores distributed throughout and a pore size range of from about 5 to about 500 microns. When the substrate is porous, the porosity of the substrate block is gradated from a first relatively low porosity region emulating or mimicking the porosity of cortical bone to a second relatively higher porosity region emulating or mimicking the porosity of cancellous bone. In a second embodiment, the substrate block is a dense solid comprised of a ceramic, metal or polymer material such as PEEK, carbon fiber reinforced polymer, PMMA, PLA or other bioresorbable polymer, or composition thereof. This dense solid substrate would then be attached to a second highly porous region emulating or mimicking the porosity of cancellous bone. Preferably, the porous region would be formed around the substrate.

In the method where a dense, solid material is used as the substrate block, the block will be externally coated with a bio-active surface coating material selected for relatively high osteoconductive and osteoinductive properties, such as a hydroxyapatite or a calcium phosphate material. The porous portion is internally and externally coated with a bio-active surface coating material selected for relatively high osteoconductive and osteoinductive properties, such as a hydroxyapatite or a calcium phosphate material. The porous region, however, may be in and of itself a bio-active material selected for relatively high osteoconductive and osteoinductive properties, such as a hydroxyapatite or a calcium phosphate material.

The thus-formed bone graft can be made in a variety of shapes and sizes to suit different specific implantation requirements. Preferred shapes include a generally rectangular or cylindrical block with a tapered cross section to suit the required skeletal anatomy. The exterior superior and inferior surfaces of the body may include ridges or teeth for facilitated engagement with the adjacent skeletal structures. Alternative preferred shapes include a generally oblong, rectangular or cylindrical block which may also include serrations or the like on one or more exterior faces thereof, and/or may have a tapered cross section for improved fit into the skeletal anatomy. A further preferred shape may include a crescent shape block which may also include serrations or the like on one or more exterior faces thereof, and/or may have a tapered cross section for improved. The bone graft may desirably include notches for releasable engagement with a suitable insertion tool. In addition, the bone graft may also include one or more laterally open recesses or bores for receiving and supporting osteoconductive bone graft material, such as allograft (donor) or autograft (patient) material.

Further alternative bone graft configurations may include a dense substrate region substantially emulating cortical bone, to define a high strength loading bearing zone or strut for absorbing impaction and insertion load, in combination with one or more relatively high porosity second regions substantially emulating cancellous bone for contacting adjacent patient bone for enhanced bone ingrowth and fusion.

The resultant bone graft exhibits relatively high mechanical strength for load bearing support, while additionally and desirably providing high osteoconductive and osteoinductive properties to achieve enhanced bone ingrowth and interbody fusion. Importantly, these desirable characteristics are achieved in a structure which is substantially radiolucent so that the implant does not interfere with post-operative radiographic monitoring of the fusion process.

In accordance with a further aspect of the invention, the bone graft may additionally carry one or more therapeutic agents for achieving further enhanced bone fusion and ingrowth. Such therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, or any other fusion enhancing material or beneficial therapeutic agent.

Other features and advantages of the invention will become more apparent from the following detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a perspective view showing the one preferred embodiment of bone graft;

FIG. 2 is a perspective view showing the load bearing portion of the device of FIG. 1 with anterior and posterior load bearing walls connected by a strut, relieved in the superior and inferior aspects;

FIG. 3 is a perspective view depicting one alternative preferred and generally rectangular bone graft;

FIG. 4 is a perspective view depicting the load bearing portion of the device of FIG. 3 with anterior and posterior load bearing walls connected by a strut, relieved in the superior and inferior aspects;

FIG. 5 is a perspective view showing still another alternative preferred form of the invention, comprising a generally oblong, rectangular bone graft;

FIG. 6 is a perspective view depicting the load bearing portion of the device of FIG. 5 with anterior and posterior load bearing walls connected by a strut, relieved in the superior and inferior aspects;

FIG. 7 is an axial view of still another alternative form of the invention, comprising a generally crescent shaped device conforming to the natural of the pelvis;

FIG. 8 is a perspective view of the device of FIG. 7, showing a porous posterior margin;

FIG. 9 is a perspective view of the load bearing portion of the device of FIG. 7, showing a anterior and lateral load bearing walls connected by a central strut, relieved in the superior and inferior aspects;

FIG. 10 is an axial view of a further preferred alternative embodiment of the invention, comprising of a generally rectangular shape with macro-pores;

FIG. 11 is a perspective view of the device of FIG. 10 showing the interconnection of the macro-pores; and

FIG. 12 is a sectional view of the device of FIG. 10 taken generally along the mid-transverse plane 12-12 of FIG. 10 of the device;

FIG. 13 is a perspective view depicting the bone graft in the inter-vertebral space;

FIG. 14 is a perspective view depicting the device in FIG. 7 in the iliac crest of the pelvis; and

FIG. 15 is a perspective view depicting the bone graft in the femur.

### DETAILED DESCRIPTION OF THE PREFERRED EMBODIMENTS

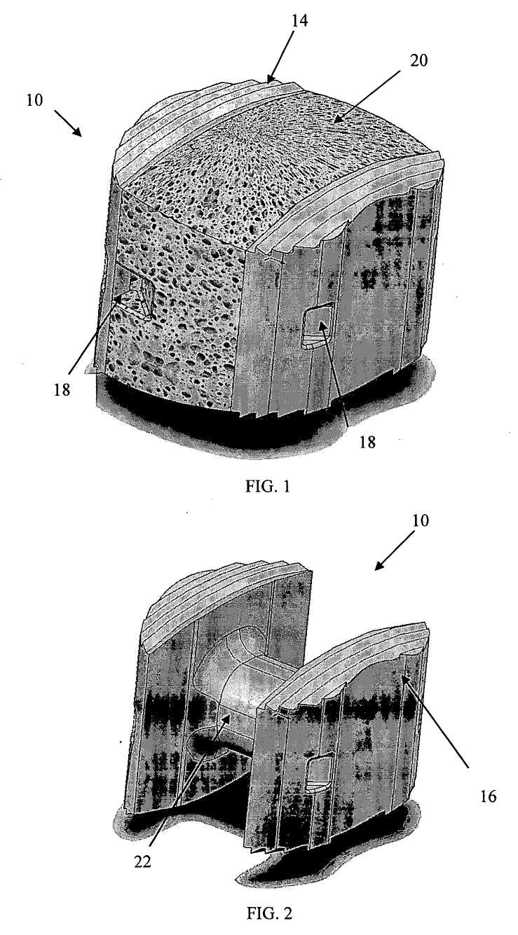

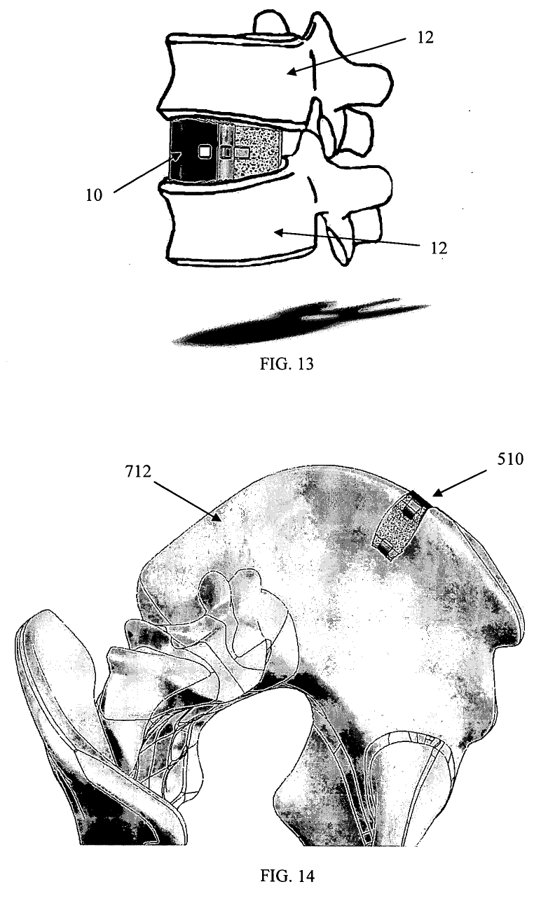

As shown in the exemplary drawings, a radiolucent bone graft referred to generally in FIGS. 1-2 by the reference numeral 10 is provided for seated implantation between a pair of adjacent patient bones 12 (FIG. 13) to maintain the skeletal tissues or structures in spaced relation while promoting interbody bone ingrowth and fusion. In general, the improved bone graft 10 comprises a bio-compatible substrate having a porous construction to define an open lattice conducive to interbody bone ingrowth and fusion, while providing a strong mechanical load bearing structure analogous to the load bearing properties of cortical and cancellous bone. This open-celled substrate is coated internally and externally with a bio-active surface coating selected for relatively strong osteoconductive and osteoinductive properties, whereby the coated substrate provides a scaffold conducive to cell attachment and proliferation to promote interbody bone ingrowth and fusion attachment. The substrate may also carry one or more selected therapeutic agents suitable for bone repair, augmentation and other orthopedic uses.

FIGS. 1-2 illustrate the improved bone graft 10 in accordance with one preferred embodiment, in the shape of a generally rectangular body having ridges formed on the top and bottom faces 14. The lateral, anterior, and posterior walls of the body having notches 18 for the releasable engagement with an insertion tool.

The preferred substrate composition comprises a relatively high strength block 16 (FIG. 2). In accordance with one preferred form of the invention, this substrate block comprises a relatively dense 16 ceramic composition having a controlled porosity and having a suitable size and shape for seated implantation, such as into the inter-vertebral space in the case of the spinal fusion cage 10. In a preferred form, the remainder of the substrate is comprised of a relatively porous ceramic 20 (FIG. 1) having an open-celled controlled porosity.

Moreover, in the preferred form, the pores are arranged with a variable porosity gradient to define a first region of relatively low or reduced porosity (less than about 5%) mimicking cortical bone structure and a second region of relatively large or increased porosity (ranging from about 30% to about 80%) mimicking cancellous bone structure. In one preferred configuration, the outer or external surfaces of the reticulated substrate block comprise the first or low porosity region for improved load bearing capacity, while the interior surfaces of the substrate block comprises the second or high porosity region mimicking cancellous bone for enhance bone ingrowth and fusion.

This high strength substrate block is surface-coated internally and externally with a bio-active organic or inorganic surface coating material selected for relatively strong osteoconductive and osteoinductive properties to provide a nutrient rich environment for cellular activity to promote interbody bone ingrowth and fusion attachment. Preferred surface coating materials comprise a resorbable material such as hydroxyapatite or a calcium phosphate ceramic. Alternative glassy (amorphous) materials having a relatively rich calcium and phosphate composition may also be used, particularly wherein such materials incorporate calcium and phosphate in a ratio similar to natural bone or hydroxyapatite. Such glassy compositions may comprise a partially or fully amorphous osteoinductive material comprising a composite of a glass and osteoinductive calcium compound, with a composition varying from about 100% glass to 100% osteoinductive calcium compound. The surface coating may also comprise autologous bone marrow aspirates.

The resultant bone graft 10 thus comprises the substrate block formed from the high strength material having bio-mimetic properties and which is nonresorbable, or slowly or infinitely slowly resorbable when implanted into the patient, in combination with the bio-active surface coating which is comparatively rapidly resorbable to promote rapid and vigorous bone ingrowth activity.

The substrate block may also advantageously be coated or impregnated with one or more selected therapeutic agents, for example, such as autologous, synthetic or stem cell derived growth factors or proteins and growth factors such as bone morphogenic protein (BMP) or a precursor thereto, which further promotes healing, fusion and growth. Alternative therapeutic agents may also include an antibiotic, or natural therapeutic agents such as bone marrow aspirates, and growth factors or progenitor cells such as mesenchymal stem cells, hematopoietic cells, or embryonic stem cells, either alone or as a combination of different beneficial agents.

The resultant illustrative bone graft 10 exhibits relatively high bio-mechanical strength similar to the load bearing characteristics of natural bone. In addition, the bone graft 10 exhibits relatively strong osteoconductive and osteoinductive characteristics attributable primarily to the surface coating, again similar to natural bone. Importantly, the bone graft 10 is also substantially radiolucent and non-magnetic, so that the fusion cage does not interfere with post-operative radiological or other imaging methods of analysis of interbody bone ingrowth and fusion.

The relatively dense, high strength portion 16 is preferably formed in a manner with which to withstand the loading of the skeletal structures. In the preferred embodiment, the anterior and posterior walls of the device are formed as part of this high strength portion. This is done to allow the high strength region to interface with the cortical portion of the adjacent skeletal body 12. Additionally, a strut 22 of the high strength material extends between the anterior and posterior walls, which beneficially provides a load bearing structure capable of withstanding impaction and insertion loading in the anterior-posterior direction. Consequently, the relatively porous portion is formed in-between the dense anterior-posterior walls and around the central strut. The porous portion thereby forms the remainder of the device, including a large region of the superior, inferior, and lateral aspects. The porous portion, being less dense in nature than the high strength regions of the device, is increasingly radiolucent, thus allowing for assessment of bone growth and bony attachment to the adjacent skeletal tissue such as adjacent vertebral bodies.

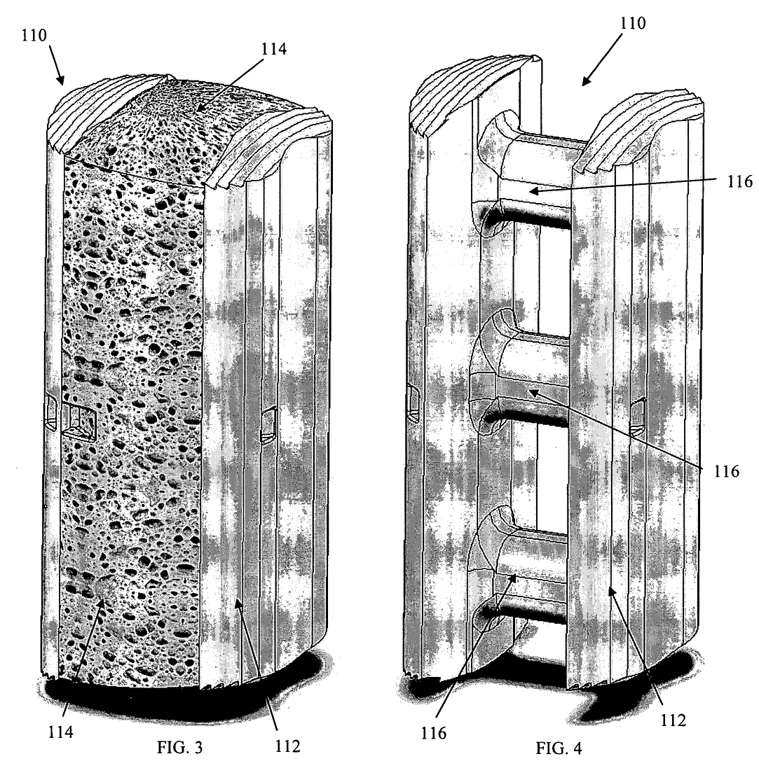

FIGS. 3-9 illustrate alternative configurations for improved bone grafts constructed in accordance with the present invention, it being recognized and understood that the bone graft can be constructed in a wide range of different geometric sizes and shapes. FIG. 3 shows a spinal fusion cage 110 having a generally rectangular shape similar to the fusion cage 10 shown and described in FIGS. 1-2, but the form is elongated, as for use in replacing an entire skeletal body. As shown, the bone graft 110 (FIG. 4) has a relatively dense structure defined by a high strength substrate block 112 (as previously described) coated with the bio-active surface coating material, but wherein the relatively dense interior structure is defined multiple struts 116 with high strength for withstanding impaction and insertion loading in an anterior-posterior direction. The multiple struts 116 additionally create interior openings which provide for lateral fluid transmission and optimize bone growth laterally through the center of the implant. FIG. 4 shows multiple dense struts, thereby demonstrating that the porous region is able to make contact with the adjacent superior and inferior vertebrae. The porous region 114 is more radiolucent than the surrounding dense portion and therefore provides enhanced visualization for analysis of bone growth and subsequent fusion with the adjacent skeletal structures. Each of the embodiments depicted in FIGS. 1-12 has a height dimension and may be tapered in shape for enhanced anatomical fit.

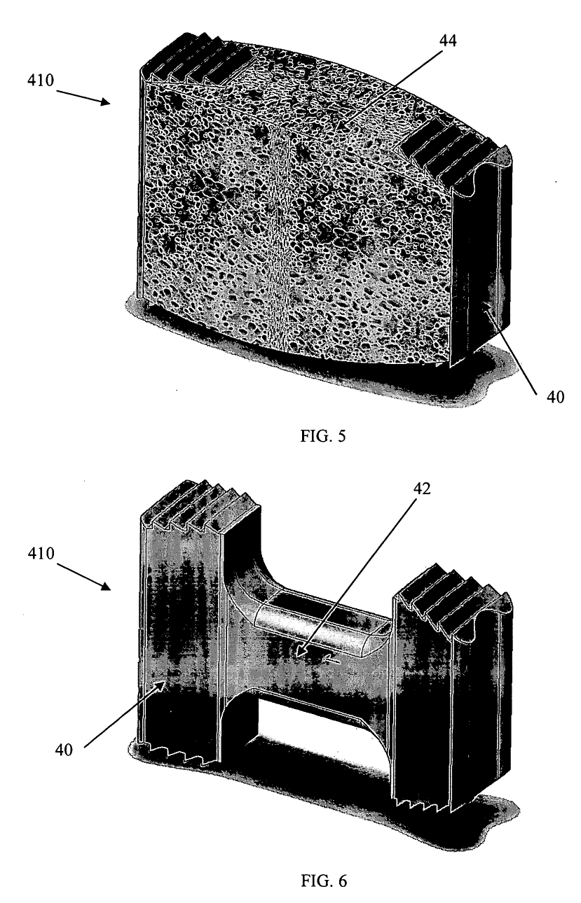

FIGS. 5-6 depicts still another alternative preferred embodiment of a generally oblong, rectangular or cylindrical geometry 410 having both a high strength, dense region 40, as well as a relatively porous region 44 for bone in-growth. This geometry would be useful for surgical approaches in which it is necessary to place two implants next to each other. More particularly, FIGS. 5-6 show a generally oblong, rectangular or cylindrical bone graft 410 having a tapered height dimension in the anterior-posterior direction. The substrate block is formed with the first region 40 of relatively low porosity substantially mimicking cortical bone to extend across the anterior and posterior faces and further to include at least one interconnecting load bearing strut 42 shown in the illustrative drawings to extend centrally in an anterior-posterior direction within the body of the substrate block. The remainder of the substrate block comprises the second portion 44 of relatively high porosity substantially mimicking cancellous bone. The harder first region 40 including the central strut 42 beneficially provides a hard and strong load bearing structure capable of withstanding impaction and insertion forces in the anterior-posterior direction without damage to the implant, while the softer second region 44 presents an exposed and large surface area for substantially optimized interknitting ingrowth and fusion with adjacent patient bone. In a spinal fusion cage application, the medial-lateral faces of the implant are advantageously defined by the softer second region 44, wherein these regions are thus exposed to traditional medial-lateral X-ray imaging for post-operative radiological analysis of the implant/bone interface. Persons skilled in the art will recognize and appreciate that alternative configurations for the load bearing strut or struts 42 may be used, such as an X-shaped strut configuration extending in a cranial-caudial direction, in combination with or in lieu of the exterior faces 40 and/or the anterior-posterior central strut as shown.

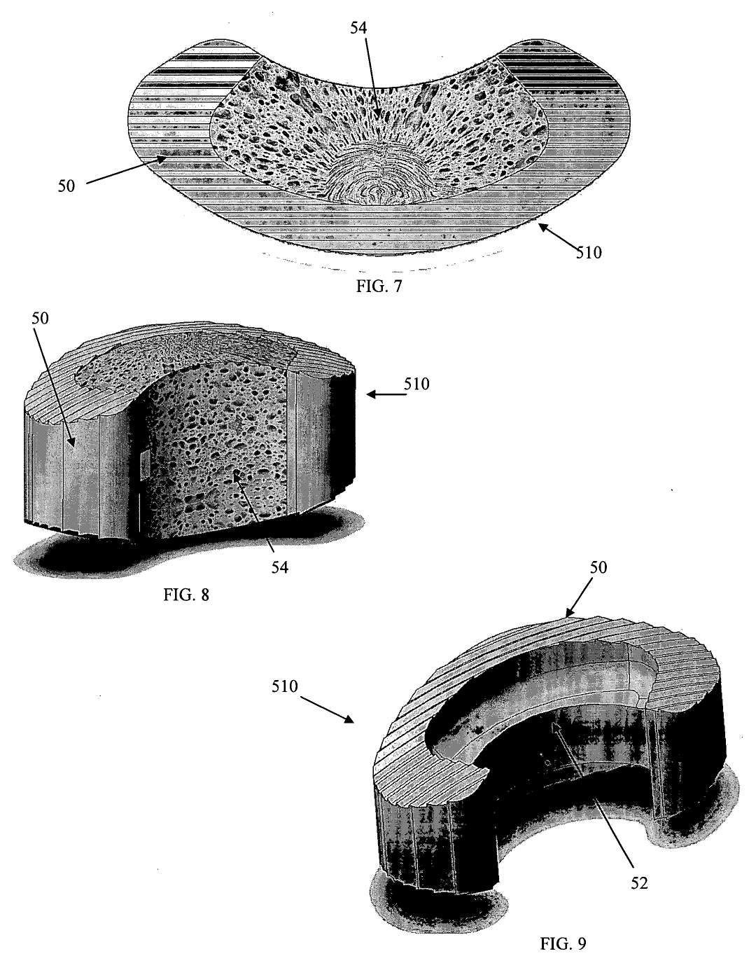

FIGS. 7-9 depict a further alternative preferred form of the invention, with a generally crescent shaped geometry 510. The substrate block is formed of a relatively dense, high strength region 50 substantially mimicking cortical bone extending along the anterior and lateral walls. The dense portion 50 once again beneficially provides a strong load bearing structure capable of withstanding loads. Also, the high-strength region 50 is located along the anterior of the substrate, thereby interfacing with the load bearing cortical bone of the adjacent skeletal body. An integral dense strut 52 extends between the dense lateral walls providing a load bearing structure for impaction and insertion forces exhibited in a lateral approach. The superior, inferior, and posterior portions of the substrate are formed with a relatively porous material 54. This provides for bone growth and increased radiolucency.

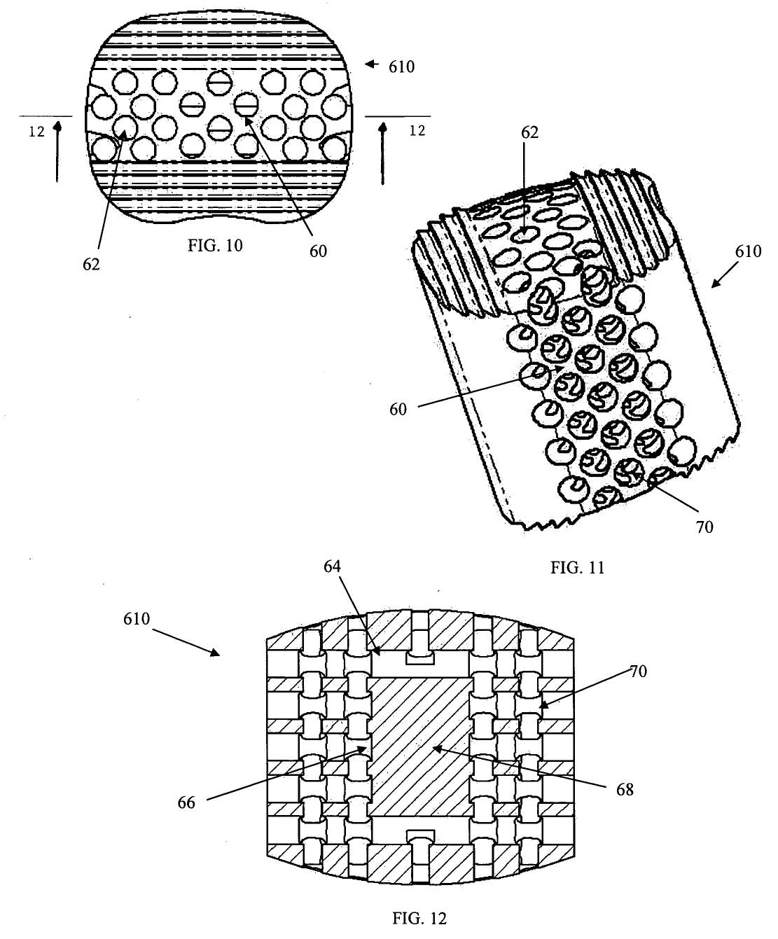

FIGS. 10-13 depict a still further alternative preferred embodiment which is formed entirely of a relatively low porosity, high-strength substrate 610. The subsequent porous structure 60 is created by drilling or boring a plurality of macro-pores 62 into the superior, inferior, and lateral faces of the device. This method allows the anterior and posterior walls to remain intact and thus be able to withstand the loading of the skeletal structures. The macro-pores are oriented in both the axial direction of the skeletal structures, as well as between the lateral walls of the device, thereby allowing bone to grow in the direction of the skeletal loading and laterally through the substrate. The macro-pores are positioned in such a manner as to allow for continuous interconnection 70, thereby creating a meshwork of pores for bony ingrowth into the device. The macro-pores extend either from one face of the device to the opposite face 64, or towards the center of the device, extended to a certain depth, and terminated therein 66. The blind macro-pores 66 in turn create a portion in the center of the device which remains solid and is therefore a load bearing strut 68 extending from the anterior wall to the posterior wall and capable of withstanding impaction and insertion loads in the anterior-posterior direction. This macro-pore method can also be utilized with geometries similar to those depicted in FIGS. 5-9, such as the oblong rectangular 410 and the crescent 510.

In all of the embodiments of FIGS. 1-12, the substrate block comprises a high strength porous ceramic as previously described, and is coated with the bio-active surface coating material, again as previously described, to enhance bone ingrowth and fusion. The substrate block may also include one or more therapeutic agents. Persons skilled in the art will recognize and appreciate that the relatively low and high porosity regions 16 and 20 shown in FIGS. 1-2 will be integrally joined by a suitable albeit relatively narrow gradient region wherein the porosity transitions there between.

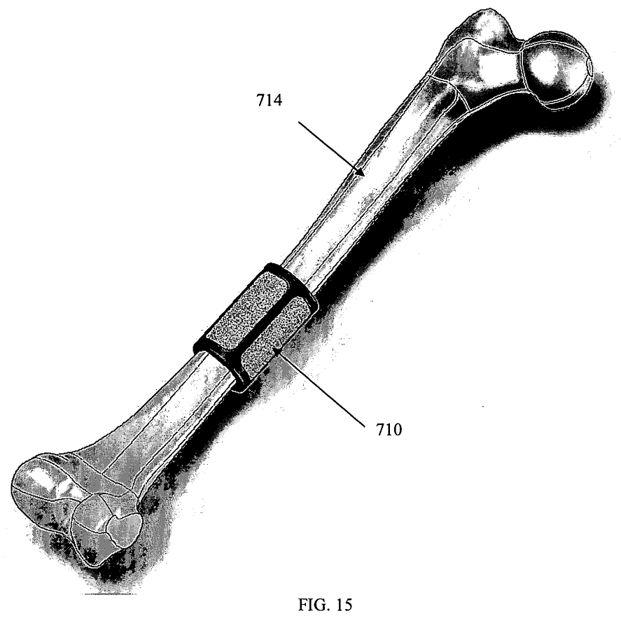

FIGS. 13-15 depict various embodiments of the bone graft in different skeletal structures. In FIG. 13, bone graft 10 is shown between two adjacent vertebral bodies 12 with the intent to enhance bone ingrowth and fusion. The bone graft 510 embodiment displayed in FIG. 14 is depicted as replacing a defect in the iliac crest 712 of the pelvic bone. In this embodiment, the defect could be a result of tumor, trauma, or surgical intervention. FIG. 15 shows a further embodiment of the bone graft 710 connecting two portions of a long bone, such as the femur 714. This embodiment of the bone graft 710 is intended to enhance bone growth and fusion while providing structural support.

The improved bone graft of the present invention thus comprises an open-celled substrate block structure which is coated with a bio-active surface coating, and has the strength required for the weight bearing capacity required of a fusion device. The capability of being infused with the appropriate biologic coating agent imparts desirable osteoconductive and osteoinductive properties to the device for enhanced interbody bone ingrowth and fusion, without detracting from essential load bearing characteristics. The radiolucent or non-magnetic characteristics of the improved device beneficially accommodate post-operative radiological or other diagnostic imaging examination to monitor the bone ingrowth and fusion progress, substantially without undesirable radio-shadowing. The external serrations or threads formed on the bone graft may have a variable depth to enable the base of the device to contact the cortical bone for optimal weight bearing capacity. In addition to these benefits, the present invention is easy to manufacture in a cost competitive manner. The invention thus provides a substantial improvement in addressing clinical problems indicated for surgical treatment of bone fractures, non-unions, weak bony tissue, degenerative disc disease, discogenic low back pain and spondylolisthesis.

The bone graft of the present invention provides at least the following benefits over the prior art:
[a] a porous osteoconductive scaffold for enhanced fusion rates;
[b] a bio-mimetic load bearing superstructure providing appropriate stress transmission without fatigue failure;
[c] a pore structure and size suitable for ingrowth and vascularization,
[d] the ability to absorb and retain an osteoinductive agent such as autologous bone marrow aspirate or BMPs;
[e] bio-inert and bio-compatible with adjacent tissue and selected for ease of resorption;
[f] fabricatable and machinable into various shapes;
[g] sterilizable; and
[h] low manufacturing cost.

A variety of further modifications and improvements in and to the bone graft of the present invention will be apparent to those persons skilled in the art. In this regard, it will be recognized and understood that the bone graft implant can be formed in the size and shape of a small pellet for suitable packing of multiple implants into a bone regeneration/ingrowth site. Accordingly, no limitation on the invention is intended by way of the foregoing description and accompanying drawings, except as set forth in the appended claims.

1. A bone graft for implantation between and fusion with adjacent skeletal tissue, comprising:
a substrate block having a first region of relatively high strength corresponding substantially with natural cortical bone and a second region of porous form corresponding substantially with natural cancellous bone.

2. The bone graft of claim 1 wherein either the first or second region of said substrate block comprises a ceramic structure formed from silicon nitride, alumina, zirconia, zirconia toughened alumina, hydroxyapatite, calcium phosphate, or composition thereof.

3. The bone graft of claim 1 wherein either the first or second region of said substrate block comprises a metallic structure formed from titanium, tantalum, stainless steel, cobalt chrome alloy, or composition thereof.

4. The bone graft of claim 1 wherein either the first or second region of said substrate block comprises a polymeric structure formed from PEEK, carbon fiber reinforced polymer, PMMA, PLA or other bioresorbable polymer, or composition thereof.

5. The bone graft of claim 1 wherein either the first or second region of said substrate block comprises a flexible material formed from silicone, polyurethane silicone, hydrogels, elastomers, or composition thereof.

6. A bone graft of claim 1 further including a bio-active and resorbable surface coating applied to said substrate block, said surface coating having osteoconductive and osteoinductive properties to promote interbody bone ingrowth and fusion attachment with the adjacent skeletal tissue.

7. The bone graft of claim 1 wherein the second region of said substrate block comprises a bio-active and resorbable material having relatively high osteoconductive and osteoinductive properties.

8. The bone graft of claim 1 wherein said the first region of said substrate block is relatively non-resorbable or resorbable at a rate substantially less than the second region.

9. The bone graft of claim 1 wherein the first region and the second region of said substrate block has a porosity ranging from about 0% to about 80% by volume, and further wherein the pore size ranges from about 1 micron to about 1,500 microns.

10. The bone graft of claim 9 wherein the first region of said substrate block has a porosity ranging from about 0% to about 50% by volume, and wherein the pore sizes range from about 1 micron to about 500 microns.

11. The bone graft of claim 9 wherein the second region of said substrate block has a porosity ranging from about 30% to about 80% by volume, and wherein the pore sizes range from about 100 microns to about 1000 microns.

12. The bone graft of claim 9 wherein the said substrate block has a variable porosity gradient substantially mimicking natural cortical and cancellous bone.

13. The bone graft of claim 9 wherein first region of said substrate block has a relatively low porosity substantially mimicking natural cortical bone, and further wherein said second region of said substrate block has a relatively high porosity substantially mimicking cancellous patient bone.

14. The bone graft of claim 9 wherein said first region has a porosity of less than about 5%, and wherein said second region has a porosity ranging from about 30% to about 80%.

15. The bone graft of claim 9 wherein said first region is generally disposed on the exterior of said substrate block, and said second region is generally disposed on interior surfaces of said substrate block.

16. The bone graft of claim 9 wherein said second region is generally disposed on the exterior of said substrate block, and said first region is generally disposed on interior surfaces of said substrate block.

17. The bone graft of claim 13 wherein said first region is generally disposed on anterior and posterior surfaces of said substrate block and further defines at least one structural load bearing strut extending through said substrate block between said anterior and posterior surfaces, said second region including an extended exposed surface area for contacting the adjacent skeletal tissue.

18. The bone graft of claim 17 wherein said second region is substantially exposed on medial and lateral surfaces of said substrate block.

19. The bone graft of claim 13 wherein said first region circumferentially surrounds and supports said second region, said second region including an extended exposed surface area for contacting the adjacent skeletal tissue.

20. The bone graft of claim 13 wherein said second region circumferentially surrounds said first region, said second region including an extended exposed surface area for contacting the adjacent skeletal tissue.

21. The bone graft of claim 13 wherein said first region comprises at least one structural load bearing strut extending through said substrate block, wherein said second region including an extended exposed surface area for contacting the adjacent skeletal tissue.

22. The bone graft of claim 1 wherein said substrate block further includes means for facilitated grasping and manipulation with a surgical instrument for implantation.

23. The bone graft of claim 6 wherein said bio-active surface coating is internally and externally applied to said substrate block.

24. The bone graft of claim 6 wherein said bio-active surface coating is selected from the group consisting of hydroxyapatite and calcium compounds.

25. The bone graft of claim 6 wherein said bio-active surface coating comprises a partially or fully amorphous osteoinductive material including a glass and osteoinductive calcium compound.

26. The bone graft of claim 6 wherein said bio-active surface coating comprises an organic coating material.

27. The bone graft of claim 26 wherein said organic coating material is selected from the group consisting of autologous bone marrow aspirates, bone morphogenic proteins, growth factors and progenitor cells, and mixtures thereof.

28. The bone graft of claim 27 wherein said progenitor cells include mesenchymal stem cells, hematopoietic cells, and embryonic stem cells.

29. The bone graft of claim 1 wherein the first region of the said substrate block is substantially radiolucent.

30. The bone graft of claim 1 wherein the second region of the said substrate block is substantially radiolucent.

31. The bone graft of claim 1 further including a therapeutic agent carried by said substrate block.

32. The bone graft of claim 31 wherein said therapeutic agent comprises a natural or synthetic osteoconductive or osteoinductive agent.

33. The bone graft of claim 1 wherein said substrate block has a rough exterior surface.

34. The bone graft of claim 1 wherein said substrate block has a ribbed exterior surface.

35. The bone graft of claim 1 wherein said substrate block has a laterally open bore formed therein, and further including an osteoconductive material supported within said bore.

36. The bone graft of claim 35 wherein said osteoconductive material comprises morselized bone graft material.

37. The bone graft of claim 1 wherein the pores formed within the second region of the said substrate block are in substantially open fluid communication sufficient to transmit fluid pressure therebetween.

38. The bone graft of claim 1 wherein the pores formed within the first region of the said substrate block are in substantially open fluid communication sufficient to transmit fluid pressure therebetween.

39. A bone graft for implantation between and fusion with adjacent skeletal tissue, comprising:
a substrate block having a relatively high strength corresponding substantially with natural cortical and cancellous bone; and
a bio-active and relatively rapidly resorbable surface coating applied to said substrate block, said surface coating having osteoconductive and osteoinductive properties to promote interbody bone ingrowth and fusion attachment with the adjacent skeletal tissue;
said substrate block being relatively nonresorbable or resorbable at a rate substantially less than said surface coating.

40. A bone graft for implantation between and fusion with adjacent skeletal tissue, comprising:
a substrate block including at least one load bearing strut having high strength structural characteristics; and
a bio-active and resorbable surface coating carried by said at least one strut, said surface coating having osteoconductive and osteoinductive properties to promote interbody bone ingrowth and fusion attachment with adjacent skeletal tissue.

41. The bone graft of claim 40 wherein said at least one strut substantially mimics the structural characteristics of natural bone.

42. The bone graft of claim 40 wherein said at least one strut is formed from a porous material.
